(19) United States
(12) Patent Application Publication
(10) Pub. No.: US 2026/0325819 A1
(43) Pub. Date: Oct. 22, 2026

(54) REPLACEMENT HOUSING FOR A HEART PUMP

(71) Applicant: Indiana University Research and Technology Corporation, Indianapolis, IN (US)

(72) Inventor: Mark D. Rodefeld, Carmel, IN (US)

(73) Assignee: Indiana University Research and Technology Corporation, Indianapolis, IN (US)

(21) Appl. No.: 19/119,253

(22) PCT Filed: Sep. 14, 2023
(86) PCT No.: PCT/US23/74201

§ 371 Date: Apr. 8, 2025

Related U.S. Application Data
(60) Provisional application No. 63/378,874, filed on Oct. 9, 2022.

(51) Int. Cl. A61M 60/135; A61F 2/06; A61M 60/216; A61M 60/35

(52) U.S. Cl. CPC A61M 60/135; A61F 2/06; A61M 60/216; A61M 60/35; A61F 2002/068

(57) ABSTRACT

Various embodiments herein pertain to implantable devices that are connected to the internal anatomy of an animal at a junction of multiple blood vessels in the circulatory system. The device includes multiple inlets and at least one outlet. The interior of the device includes a flow diverter to organize the internal flow paths from inlet to outlet.

### FIELD OF THE INVENTION

The present inventions pertain to methods and apparatus for implanting a device in an animal, and more particularly for implantation of a diverting device in a junction of blood vessels in a circulatory system.

### BACKGROUND OF THE INVENTION

Various embodiments of the present invention pertain devices useful with a cavopulmonary assist device, also known as a Fontan pump, to provide a subpulmonary power source in a univentricular Fontan circulation, although some embodiments are not so limited and apply to other pumping devices or implanted devices. The pump is implanted in-series with the circulation as a permanent implant. As such, it has potential to obstruct cavopulmonary blood flow if the device were to ever fail. The device is however designed so that it will not obstruct passive flow at 0 RPM.

If a device fails and cannot be made operational, then the question arises as to what should be done with the failed device? Options are to surgically remove the failed device, and either 1) replace it with a new one; or 2) replace it with a passive flow diverter. In some cases, it may be preferable or clinically indicated to follow option 2. In this case, the failed device and hardware can be removed, and a passive flow diverter can be used to replace it. This component would be optimized for passive flow, with least possible pressure loss and thrombogenic potential. Further, provision of a clinical solution or exit strategy for failed device removal will likely be required by regulatory agencies, as there is no precedent for leaving a failed device permanently implanted.

### SUMMARY OF THE INVENTION

In one embodiment, the present invention pertains to a housing with a flow diverting member that is implantable in the junction of a circulatory system of an animal. This housing and flow diverter provide improved blood flow characteristics for the multiple blood vessels the are in fluid communication at the junction.

In yet another embodiment, the present invention pertains to an assembly of a housing and a flow diverter that replace a ventricular assist device or other blood pump that was located at a junction of multiple blood vessels. After the VAD or pump is removed from the patient, the assembly may provide improved flow characteristics within the junction, in some cases reducing turbulence that could otherwise damage blood cells, or in other cases improving the throughput of blood within the junction. Although an assembly has been referred to, it is understood that the assembly can be either a combination of multiple separate pieces, or a single piece, including single pieces fabricated by molding or casting. by computer aided deposition, or by 3D printing, in each case with or without subsequent machining.

In yet another embodiment, the assembly of housing and flow diverter are placed at a junction in the circulatory system that includes two blood vessels providing blood with one blood vessel that carries away the combined flow. The assembly provides improved flow characteristics from the 2 inflowing vessels to the 1 outflowing vessel. In some embodiments this junction formerly was in fluid communication with a pump or other device prior to the implantation of the assembly.

Still further embodiments pertain to an assembly of a housing and internal flow diverter that are placed at a junction of 2 inflowing vessels and 2 outflowing vessels that were arranged during a Fontan procedure. This assembly may be implanted prior to implantation of a pump at the Fontan junction, such as in cases in which the pump is implanted at a later date either as a matter of routine scheduling or as a matter of a complication. The assembly may also be implanted after a pump has been removed from the junction.

in some embodiments, the flow diverter is adapted and configured with sufficient symmetry such that the portion of the diverter receiving inflow from each inlet is adapted and configured to direct substantially equal outflow to the portion of the diverter that directs fluid to each outlet. In some embodiments this substantially equal apportionment to the outlets is achieved with one or two planes of symmetry of the flow diverter relative to the housing, even if the housing has less symmetry or no symmetry.

Yet another embodiment pertains to a preferably passive flow diverter that is interchangeable with an active, motorized pump. Preferably, it would have no moving parts and no electrical components. It would be designed for optimal flow properties to minimize pressure loss and thrombogenic potential.

In one embodiment, the present invention pertains to a pump that is implantable within the circulatory system of an animal. The device is connected to the circulatory system by one or more graftable conduits that connect to vascular tissue. These vascular connections can be performed by suturing. However, the conduit is also in fluid communication with the device at inlet and outlet ports that cannot be sutured to the harder surfaces of the device. Instead, each of the conduits includes a rigid or semi-rigid connection that can be mechanically coupled to a complementary connection on the device body. In some embodiments, these connections are circular flanges held together by a clamp.

Still further embodiments of the present invention pertain to an implantable housing having an external shape the same as an implantable blood pump. The housing is useful for implanting separate ducts grafted into the circulatory system. The proper installation of the ducts can be verified with the housing prior to implantation of the pump.

Yet other embodiments of the present invention pertain to the suturing connection of separable fluid ducts into the anatomy of an animal, with subsequent attachment of an implantable pump into the animal, the pump being in fluid communication with the fluid ducts and the animal circulatory system.

Various embodiments of the present invention provide one or more of the following aspects, each provided as a non-limiting example:
Seamless PTFE connector on each graftable conduit.
4 grafts sutured to each vessel; reinforced or non-reinforced graft; length specified by surgeon.
If the graft connection is unsatisfactory, the graft can be replaced without incurring the cost of an entire pump.
The pump body is not in the way of the surgeon during suturing of the graftable conduits.
A “blank” housing can be used to assess spacing of the conduits.
Pump can be inserted after graft patency is assured.
Use quick-connect tool to attach the 4 grafts to the pump.
Facilitates surgical implantation, improves accuracy of implantation, reduces risk of adverse events, reduces risk of accidental damage to a high-precision implantable medical device.

It will be appreciated that the various apparatus and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

Some of the figures shown herein may include dimensions. Further, the figures shown herein may have been created from scaled drawings, scaled models, or from photographs that are scalable. It is understood that such dimensions, or the relative scaling within a figure, are by way of example, and not to be construed as limiting unless so stated in a claim.

FIG. 1A is a side view of a portion of a circulatory system of an animal with the circulatory system being partly modified in preparation for a Fontan procedure.

FIG. 1B is a view of the circulatory system of FIG. 1A showing the circulatory system as prepared for bypassing operation.

FIG. 1C is a view of the circulatory system of FIG. 1B prior to implantation of a pump in the Fontan junction.

FIG. 1D shows the circulatory system of FIG. 1C with the pump implanted.

FIG. 2A is a close-up of a portion of a circulatory system that is prepared for insertion of a pump.

FIG. 2B shows the circulatory system of FIG. 2A with a pump implanted, the pump being shown in partial cutaway view according to one embodiment of the present invention.

FIG. 3 is an exploded view of an implantable pumping assembly according to another embodiment that includes a central blood pressure-boosting device and four graftable conduits.

FIG. 4 is a cross sectional representation of an implanted pumping assembly connected by four graftable conduits to a circulatory system.

FIG. 5A shows a perspective photographic representation of four conduit blanks prior to their conversion to a graftable configuration.

FIG. 5B shows one of the conduit blanks of FIG. 5A being modified to a graftable configuration.

FIG. 6A shows a perspective photographic representation of a clamp assembly according to one embodiment of the present invention.

FIG. 6B shows a perspective photographic representation of a locking device according to one embodiment of the present invention.

FIG. 6C shows a perspective photographic representation of a clamp assembly according to one embodiment of the present invention.

FIG. 6D shows a method of coupling two flanged attachments together.

FIG. 7A shows a variety of flange configurations useful for alignment of the conduits and housings in some embodiments of the present invention.

FIG. 7B shows another variety of flange configurations useful for alignment of the conduits and housings in some embodiments of the present invention.

FIG. 8 is a cross-sectional view of an implanted replacement housing.

FIG. 9A is an orthogonal view of the apparatus of FIG. 9B.

FIG. 9B is a side elevational view of a portion of the apparatus of FIG. 8.

FIG. 9C is an orthogonal view of the apparatus of FIG. 9B.

FIG. 10 is a cross-sectional view of an implanted replacement housing according to another embodiment of the present invention.

FIG. 11A is an orthogonal view of the apparatus of FIG. 11B.

FIG. 11B is an external, side elevational view of the housing of FIG. 10.

FIG. 11C is an orthogonal view of the apparatus of FIG. 11B.

### ELEMENT NUMBERING

The following is a list of element numbers used with all of the embodiments, and at least one noun used to describe that element. It is understood that none of the embodiments disclosed herein are limited to these nouns, and these element numbers can further include other words that would be understood by a person of ordinary skill reading and reviewing this disclosure in its entirety.

| | |
|---|---|
| 10 | circulatory system |
| 12 | Fontan junction |
| 18 | sutures |
| 20 | implantable pumping assembly |
| .1 | direction of flow |
| 22 | centerline |
| 26 | conduit, electrical power |
| 30 | housing |
| 32 | body |
| .1 | wall |
| .2 | interior |
| .3 | exterior |
| .4 | chamber |
| .5 | side |
| 34 | inlet; fluid connection |
| .1 | connection feature |
| .2 | sealing face or compression face |
| .3 | periphery |
| .4 | bearing support, strut |
| .3 | cross sectional area |
| 38 | outlet; fluid connection |
| .1 | connection feature |
| .2 | sealing face or compression face |
| .3 | periphery |
| .4 | gap |
| 40 | pump; impeller |
| 42 | pumping surface |
| 44 | outlet, cooling flow |
| 50 | motor |
| 52 | shaft |
| 54 | bearings |
| 60 | graftable conduit |
| .1 | conduit, pump inlet |
| .2 | conduit, pump outlet |
| .3 | conduit, pump inlet |
| .4 | conduit, pump outlet |
| 62 | body |
| .1 | end |
| .2 | lumen |
| .3 | flexible portion |
| .4 | rigid portion |
| 64 | inlet |
| .1 | housing connection end; means for coupling to the |
| | housing; flange |
| .12 | sealing face or compression face |
| .14 | periphery |
| .2 | anatomical connection end; means for coupling to a |
| | circulatory system |
| .3 | inlet flow area |
| 68 | outlet |
| .1 | housing connection end; means for coupling to the |
| | housing; flange |
| .12 | sealing face or compression face |
| .14 | periphery |
| .2 | anatomical connection end; means for coupling to a |
| | circulatory system |
| .3 | outlet flow area |
| 70 | clamp assembly |
| 72 | channel |
| 74 | lock |
| 75.1 | threads |
| 75.2 | bolt |
| 76 | seal |
| 80 | replacement housing assembly |
| 82 | housing |
| 86 | flow diverter |
| .1 | leading edge |
| .2 | trailing edge |
| .3 | central body |
| .4 | flow surface |
| .5 | minimum flow cross sectional area; gap |
| 88 | support strut, flow diverter |
| .1 | leading edge |
| .2 | trailing edge |

### DETAILED DESCRIPTION OF ONE OR MORE EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates. At least one embodiment of the present invention will be described and shown, and this application may show and/or describe other embodiments of the present invention, and further permits the reasonable and logical inference of still other embodiments as would be understood by persons of ordinary skill in the art.

It is understood that any reference to “the invention” is a reference to an embodiment of a family of inventions, with no single embodiment including an apparatus, process, or composition that should be included in all embodiments, unless otherwise stated. Further, although there may be discussion with regards to “advantages” provided by some embodiments of the present invention, it is understood that yet other embodiments may not include those same advantages, or may include yet different advantages. Any advantages described herein are not to be construed as limiting to any of the claims. The usage of words indicating preference, such as “various embodiments” or “preferably,” refers to features and aspects that are present in at least one embodiment, but which are optional for some embodiments, it therefore being understood that use of the word “preferably” implies the term “optional.”.

Although various specific quantities (spatial dimensions, temperatures, pressures, times, force, resistance, current, voltage, concentrations, wavelengths, frequencies, heat transfer coefficients, dimensionless parameters, etc.) may be stated herein, such specific quantities are presented as examples only, and further, unless otherwise explicitly noted, are approximate values, and should be considered as if the word “about” prefaced each quantity. Further, with discussion pertaining to a specific composition of matter, that description is by example only, and does not limit the applicability of other species of that composition, nor does it limit the applicability of other compositions unrelated to the cited composition.

Some of the inventions disclosed herein relate to a cavopulmonary assist device, also known as a Fontan pump; this disclosure describes a sub-component of this technology. It is logical to expect that an implantable blood pump may fail at some point, and alternative strategies will be necessary to deal with it clinically. If a pump were to fail, it is designed so that it will not obstruct flow in the Fontan circuit. To manage this scenario clinically, a decision will need to be made as to several options: 1) replace with a new pump; 2) replace with a blank housing; 3) leave the existing pump in place forever.

A clinician will need to have an option to replace the failed pump with a blank pump housing or a passive-flow optimized flow diverter as a remedy to removal of a failed pump to optimize the long-term health of the patient. Having an available replacement alternative will also be important and likely mandatory to regulatory agencies to ensure device safety. It will be challenging for regulatory agencies to feel comfortable with approval of leaving a failed device in place forever; there is no precedent. Some embodiments of the present invention relate to the pump replacement component, designed for passive flow optimization, and preferably with no mechanical or moving components. Further, the non-functional replacement component is envisioned to be interchangeable with the motorized pump so that exchange can be performed easily and quickly.

This disclosure also relates to methods and apparatus for surgical implantation. When a blood pump is surgically implanted, it is attached to blood vessels or the heart. Surgeons typically suture these attachments to the appropriate anatomic structures, and may then separately attach the pump using a coupling method if the pump is so designed.

In the case of using a pump in a Fontan arrangement of the circulatory system, the space for suturing is critically limited because the pump is placed in situ in an anatomically tight space adjacent to the aorta, trachea, etc. Four blood vessel connections are required in the case of a Fontan procedure, although the present invention also contemplates implantations in “Y” shaped junctions having two inlets and a single outlet, and further contemplates implantations of “Y” shaped junctions having two outlets and a single inlet, and further contemplates any implantable device having at least one fluid connection. Because of the bulk of the pump, it may be difficult to suture each of the connections without being constrained by lack of sufficient space to safely and successfully complete the anastomoses (connections).

Various embodiments of the present invention contemplate a novel method of using pre-formed suturable grafts (by way of example only, expanded polytetrafluorethylene, ePTFE, commonly known as Gore-tex®) to facilitate blood vessel anastomosis. Rigid pre-formed ends of the grafts (PTFE ends, not ePTFE ends) are seamlessly integrated to the flexible portion of the graft and allow for secure connection to the pump body with minimal seam or irregular flow surface. However, yet other embodiments contemplate graftable ducts that are assemblies of multiple components, such as by way of example only separate flexible ducts and less flexible housing connections joined by methods such as ultrasonic or other welding, as non-limited examples.

Each of the 4 grafts can then be connected to the pump body using a simple quick connect method (by way of example only, a flange clamp) to securely attach the rigid pre-formed ends of the grafts (i.e. flanges) to the pump. This novel method in some embodiments simplifies surgical implantation. Further, it can allow for pump exchange/upgrade to be performed more rapidly and with less health risk if pump change-out is ever necessary. It is further understood that although the use of ePTFE and PTFE is shown and described, various embodiments contemplate the use of any material.

This disclosure describes method and apparatus for surgical implantation of a blood pump. The pump uses in some embodiments 4 connections to blood vessels in a tight anatomic space. Using flexible PTFE grafts with rigid pre-formed connector ends (for connection to the pump housing) facilitates blood vessel sutured connections before the pump body is implanted. A ‘dummy’ (non-functional) pump sizer can be used by the surgeon to judge graft length and spacing. This dummy pump in some embodiments is a housing that preferably has the general form of the final implantable device. This dummy device can have only the external connections, although in other embodiments the dummy device can have an open interior chamber, such as a housing that is internally formed to support the final implantable pump or other components.

The grafts incorporate a flexible section of ePTFE that can be sutured to the patient anatomy. After the 4 grafts are properly placed, and vascular patency is assured, the pump can be put in position and connected to each of the 4 grafts preferably using a simple connector tool. This method also facilitates subsequent pump exchange, if ever needed, by allowing the surgeon to replace only the pump body without having to perform a delicate, complex, and risky dissection to perform new sutured connections to vascular structures that are located in critical spaces.

Flexible ePTFE grafts with rigid pre-formed PTFE connector ends can be used for suture connection to target blood vessels without having any obstruction from the body of the pump. The grafts can be tailored to the length and diameter needed, and can be sounded after suturing to ensure a widely patent connection. Once complete, the pump can be easily placed into position and attached using a simple clamp tool.

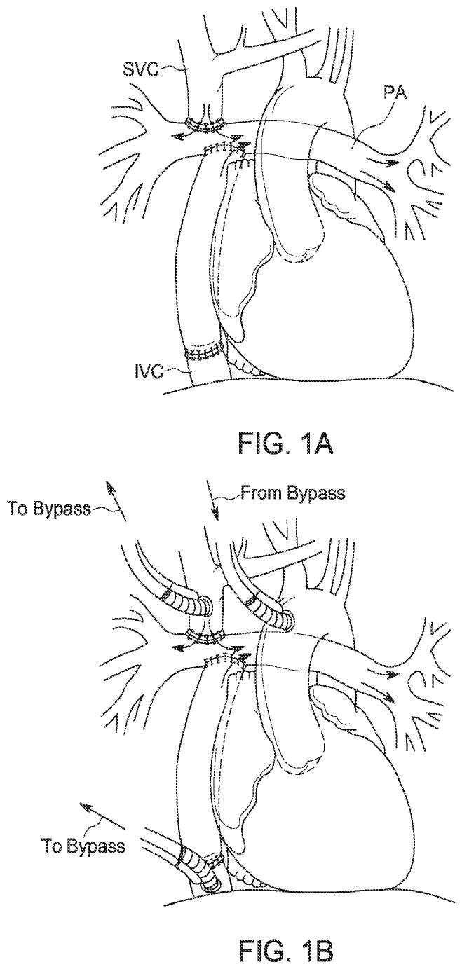

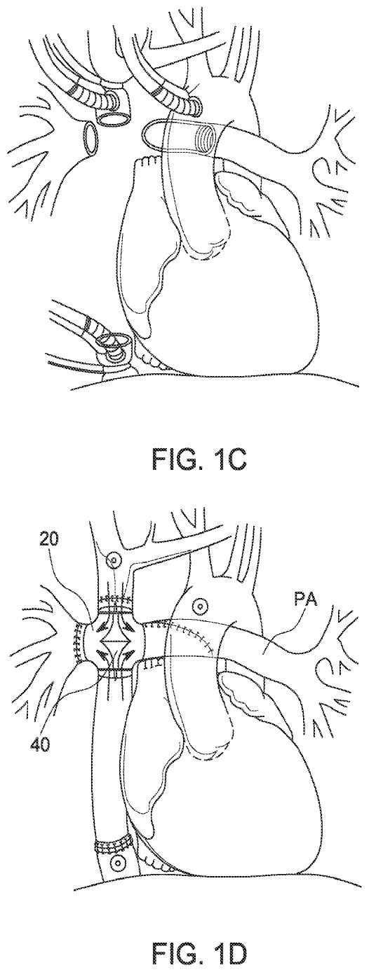

FIGS. 1A, 1B, and 1C each show a heart and the surrounding circulatory system being modified to include a Fontan junction. In FIG. 1D the junction is provided with an implantable pumping assembly 20 that is adapted and configured to provide a boost in blood pressure of less than about 15 mm Hg.

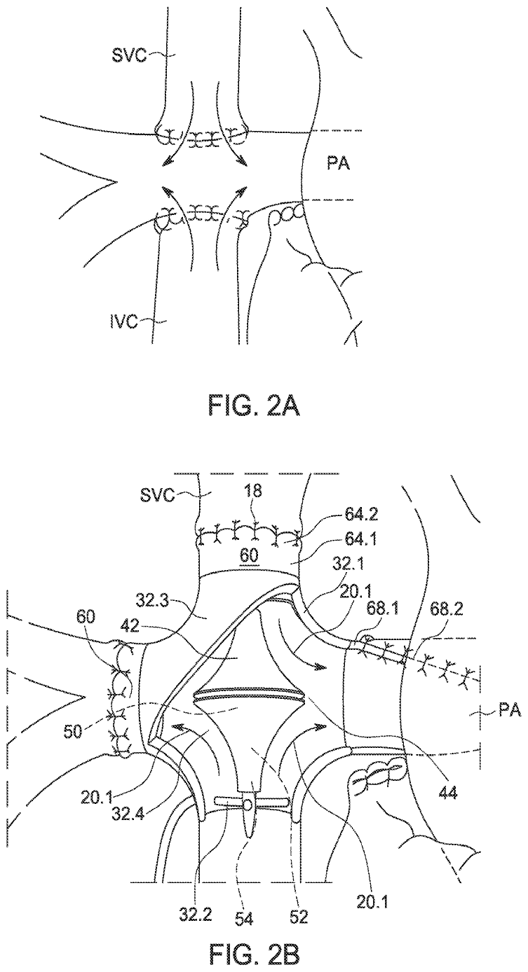

FIG. 2A is a close-up schematic representation of a Fontan junction, in which the superior vena cava (SVC) and the inferior vena cava (IVC) are modified to provide blood to the pulmonary artery (PA). FIG. 2B shows this same junction after an implantable pumping assembly 20 has been placed at the junction.

FIG. 2B shows a pumping assembly 20 in partial cross section. A rigid housing 20 supports a rotatable impeller 40 that is supported by bearings 54 and one or more struts 34.2. A motor 50 (not shown) is located within the impeller 40. The motor provides an electromagnetic field that spins the rotor. Blood flowing around the exterior of the rotor is viscously propelled by the rotor to exit the SVC or IVC and flow into the pulmonary artery. Also shown is an outlet 44 that provides an outlet for flow used to cool the motor 50.

FIG. 2B shows that the implantable pumping assembly 20 includes a housing 30 which in some embodiments (such as the one shown) includes a pair of opposing inlets 34 through which the rotor assembly is supported by struts 34.2. Blood is propelled to flow to a pair of outlets 38. Housing 30 comprises a body 32 having walls 32.1 that interconnect the inlets and outlets. The interior 32.2 of the housing is substantially open, and defines a chamber 32.4 in which the rotor is free to operate.

FIG. 2B further shows that each inlet 34 and each outlet 38 include a graftable conduit 60 that provides a connection interface between housing 30 and the associated vascular system. In one embodiment of the present invention, each conduit 60 comprises a flexible material such as ePTFE material. Each graftable conduit 60 includes a portion 64.1 (at the body inlets) and 68.1 (at the body outlets) that is adapted and configured to connect to the rigid or semi-rigid housing body 32. Each conduit 60 also includes, at the opposite end of the respective conduit, a portion 64.2 (inlet) or 68.2 (outlet) that is adapted and configured for connection to the circulatory system. As shown, each of the ends 64.2 and 68.2 are fabricated from a material (such as ePTFE) that is adapted and configured to be coupled to the circulatory system by sutures 18. The connections 64.1 and 68.1 can be made mechanically to body 32 in any manner.

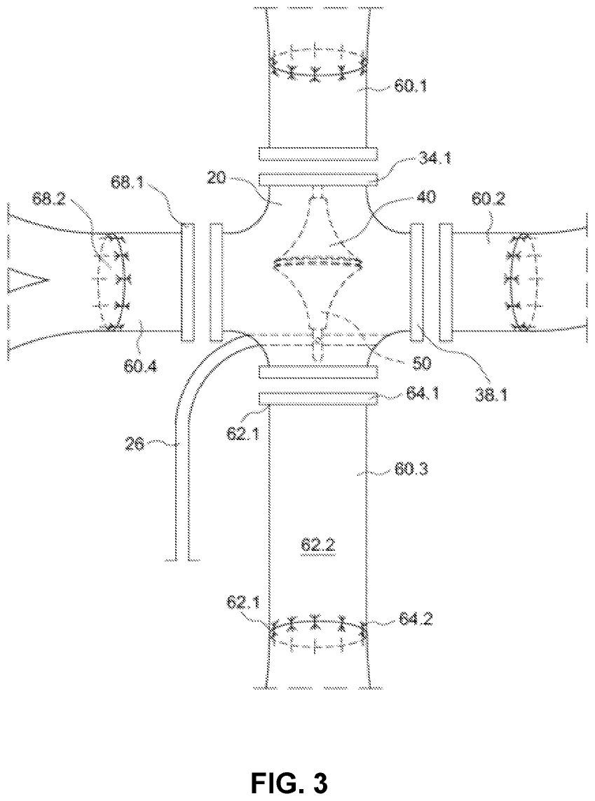

FIG. 3 is an exploded, perspective, line drawing of an installed implantable pumping assembly 20 according to another embodiment of the present invention. Placed centrally within FIG. 3 is an implantable pumping assembly 20 that operates substantially the same as the assembly 20 discussed with regards to FIG. 2B. It can be seen that a plurality of separable, graftable conduits 60 surround the pumping assembly 20, with a pair of conduits 60.1 and 60.3 arranged proximate to corresponding inlet connection features 34.1 of body 32. These inlet conduits 60.1 and 60.3 are shown at the top and bottom of FIG. 3. Also shown in FIG. 3 is a pair of separable, graftable conduits 60.2 and 60.4 shown on the right and left sides of body 32, and located proximate to the outlet connection features 38.1 of body 32. After the conduits are connected to the body, as will be described later, the lumen of each conduit is in fluid communication with the interior of body 32.

Various embodiments of the present invention contemplate that the graftable conduits 60 can be of different sizes in order to achieve proper flow conditions within the circulatory system. For example, the inner diameters of conduit 60.1 and 60.3 in some embodiments are 18 mm and 20 mm, respectively. The inner diameters of outlet conduits 60.2 and 60.4 are preferably 16 mm and 16 mm, respectively. It is further noted that each conduit can have a custom length from the housing connection end (64.1 or 68.1) to the anatomical connection end (64.2 or 68.2), as can be seen in comparing the schematic representations of inlet conduits 60.1 and 60.3.

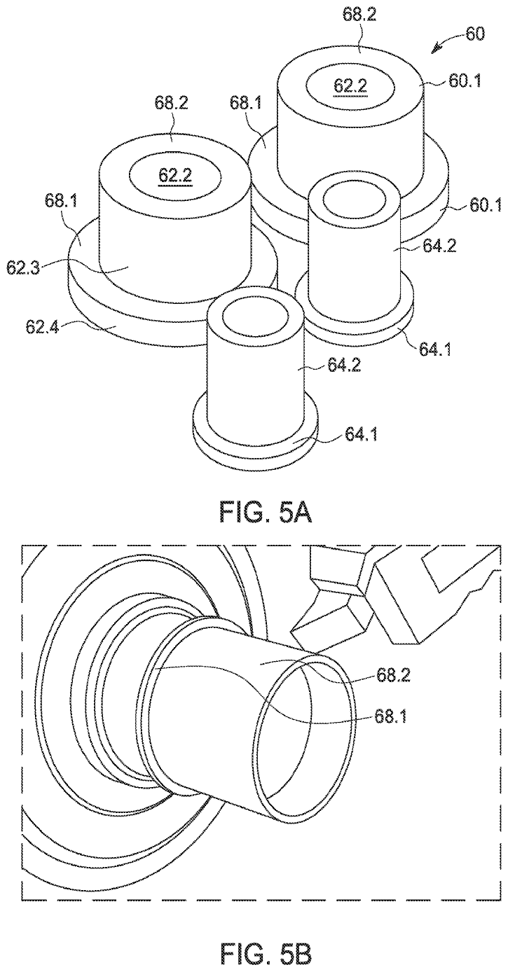

Further details of a graftable conduit 60 can be seen with reference to FIGS. 5A and 5B. FIG. 5A shows a plurality of conduit blanks. In some embodiments, each conduit 60 includes a body 62 having a pair of opposing ends 62.1 and defining a lumen 62.2 therebetween. Each conduit 60 preferably includes a flexible portion 62.3 and a rigid portion 62.4. FIG. 5B illustrates the conversion of one of the blanks 60 of FIG. 5A being converted into a graftable conduit 60 by stretching or otherwise modifying the PTFE into expanded PTFE (ePTFE).

In some embodiments, each conduit 60 is preferably a unitary construction without seams so as to eliminate potential failure sites. A conduit blank such as those shown in FIGS. 5A and 5B can be provided with a controlled temperature gradient with the use of appropriate heat sources and heat sinks, and fabricated to have a less rigid lumen section integrated with a more rigid flange section. As one example, the portion of the blank to be formed into flexible portion 62.3 can be exposed to a localized source of heat. The portion of the blank to be formed into rigid portion 62.4 can be exposed to a localized source of active cooling, or to a heat sink. The blank can then be formed such as by drawing or other means to stretch and thin the lumen section 62.3 and preferably expand the native PTFE. It may be helpful during final curing to control the rate of cooling after the conduit has been initially formed, especially for purposes of maintaining appropriate density and shortening. In some embodiments, the flange portion 62.4 remains with the native material properties.

Still further, the stretch ratio applied to the blank can be varied along the axial length of the blank, with the wall of the flexible portion preferably being thinner than the wall that is proximate to the rigid (flanged) section so as to result in an acceptable stress profile in the geometry transition from the cylindrical wall of the conduit to the thicker flange.

Preferably, the finished conduit will have a flexible walled section that is softer, more compressible, more flexible, and accommodate greater tissue growth (endothelization) than the flanged portion. Likewise, the rigid, flanged portion of the conduit will be stiffer, less compressible, less flexible, and accommodate less endothelization than the flexible portion.

Although what is shown and described are conduits 60 that comprise PTFE and ePTFE, various embodiments of the present invention contemplate conduits 60 that can be fabricated from any kind of biocompatible material. Preferably, each conduit 60 is of a unitary, integrated structure comprising a rigid or semi rigid housing connection end 68.1 (for an outlet) that is integral with a flexible, suturable anatomical connection end 68.2. In one embodiment, a graftable conduit is fabricated from a PTFE blank that is rigid or semi-rigid.

The rigid end 68.1 is preferably modified to include a flange at the housing connection end. Moving distally from that housing connection end, the cylindrical selection of the blank is then appropriately modified to ePTFE, which results in the cylindrical section being flexible as compared to the flange end. With this flexibility, along with the reduced wall thickness of the anatomical connection end, the anatomical connection end becomes adapted and configured for connection by suture to the circulatory system.

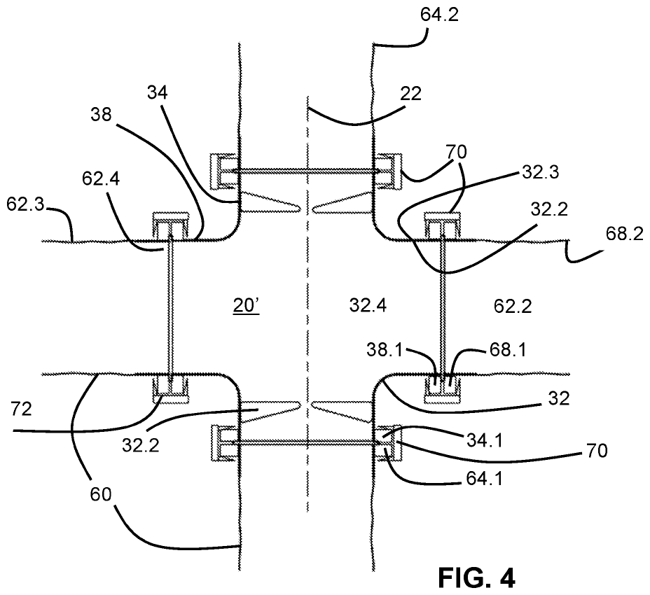

FIG. 4 shows a cross sectional schematic representation of a mock-up or dummy implantable assembly 20′ that is coupled to a plurality of conduits. As shown in FIG. 4, a body 32 (not including an impeller 40 or motor 50) is shown interconnected to a plurality of conduits (as shown exploded from the body in FIG. 3). Some embodiments of the present invention include the use of an empty housing body as a means to perform a fit check and verify the proper placement of the graftable conduits 60. After the correct placement has been demonstrated, the dummy pumping assembly 20′ can be easily removed and replaced with the functioning pumping assembly 20, in a manner as will be described.

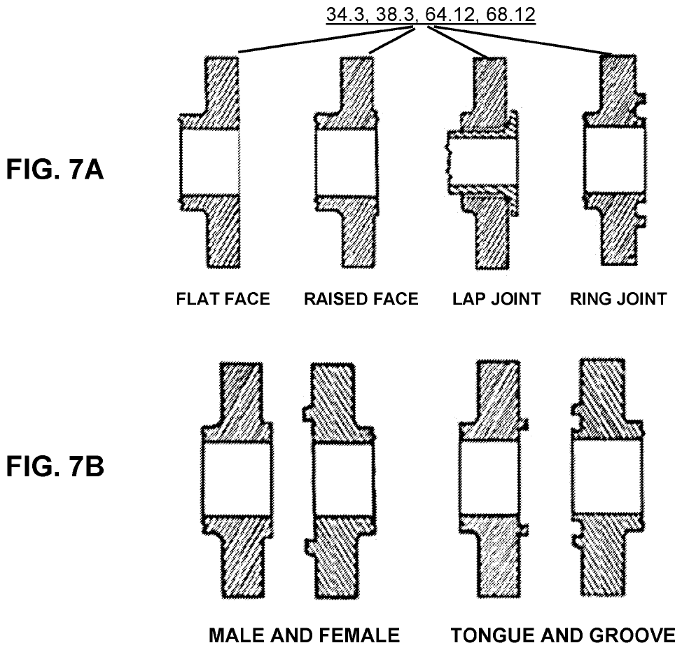

FIG. 4 shows that the housing connection ends 64.1 (inlet) or 68.1 (outlet) each preferably include a preferably flat, outwardly extending face or flange that is placed in abutting relationship with the substantially flat face of the respective housing connection feature 34.1 (inlet) or 38.1 (outlet). Although what has been shown and described is flange to flange interface that is substantially flat, further embodiments of the present invention contemplate the pairing of any structure of conduit to body interfaces other than flanges, and any manner of interface between the faces of the housing and conduit interfacing structures, including structures adapted and configured to provide locating features or sealing features. Various types and configurations of flange locating features are shown in FIGS. 7A and 7B. Further, it is understood that the flange to flange mating interface can include accommodations (such as a groove) for a seal.

Referring again to FIG. 4, after the housing flange and the conduit flange are placed in abutting relationship to each other, a clamp 70 is placed to substantially surround the outer diameter of the pair of flanges. Preferably, this clamp 70 extends substantially around the entirety of the periphery of the mating flanges. In some embodiments, the clamp 70 includes a channel 72 that is adapted and configured to provide compression of the flanges against each other. In the embodiment shown, channel 72 includes one or more angled internal surfaces that provide the face to face compression as the clamp 70 is tightened around the periphery of the connect flanges. In this embodiment, a wedging action occurs as the open and loosened clamp 70 is tightened into place. A non-limiting example of a clamp useful in some embodiments is similar to a Marmon clamp. FIG. 4 further show an example of a gap 38.4 between a housing flange and an external surface of the housing. This gap permits sufficient clearance to prevent interference between the clamp and the housing, and in some embodiments also provides sufficient clearance to prevent interference between adjacent clamps.

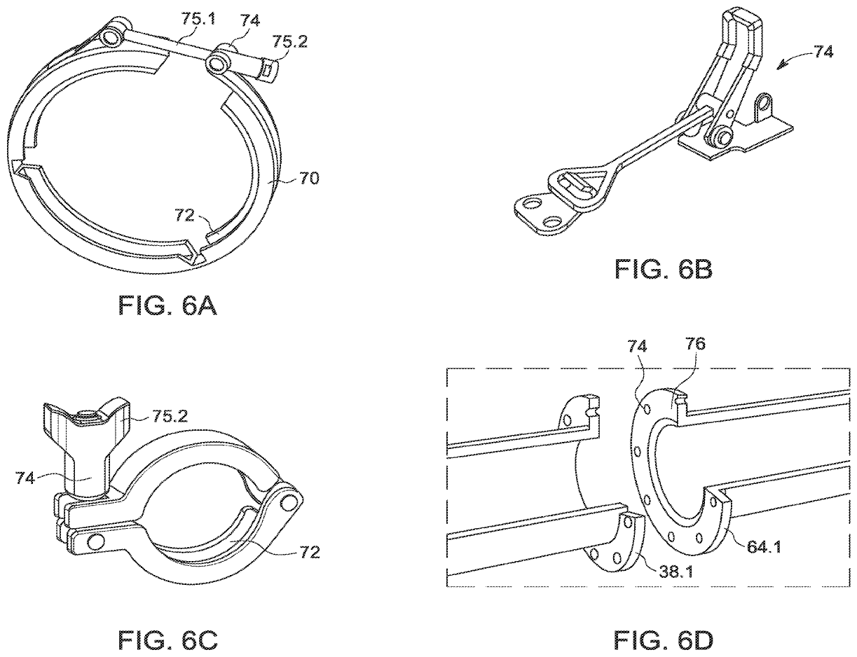

FIG. 6A shows an example of one such clamp. Clamp 70 of FIG. 6A includes a channel 72 provided on a multi-section split ring, with the clamp including a locking mechanism 74 located at the split. It can be seen that at the split there is a pivotal bolt having a threaded section 75.1 that extends through an eye on the opposite side of the split. As the nut 75.2 is tightened on the threaded portion, the extended, opened diameter of the lock is reduced to a locking configuration, from which the channel 72 applies compression to the mating flange faces. FIG. 6C shows yet a different clamping assembly 70 that can be used to apply compression to the flange faces. FIG. 6B shows yet a further device for pulling together the split ends of a clamp. It is understood that each end of the lock 74 of FIG. 6B is shown by itself, without the channeled split ring. Still further, FIG. 6D shows another manner of providing compression between two flange faces by way of a plurality of nuts and bolts (not shown) or other fasteners that extend between mating holes in the flanges. In some embodiments, a seal 76 such as a gasket or O-ring is further provided between the flange faces.

FIGS. 7A and 7B show a variety of flange configurations useful in various embodiments of the present invention. It is understood that each of the flange configurations shown in these two figures represent cross sectional depictions through the duct centerline of flanges having preferably circular peripheries.

The flange identified as “flat face” is the same as the flange faces depicted in FIG. 4. These faces are substantially flat and smooth, and preferably have faces that are generally orthogonal to either the duct flange periphery or the duct centerline. These faces can be brought together in a sealing configuration with any of the means for compressing flange faces shown in FIG. 3, 4, or 6. It is further understood that the means for compression shown in FIG. 6D does not include a ring surrounding the periphery of the flange, but rather depicts flange faces in which at least one flange contains a through hole that aligns with a through hole (or a threaded hole) in the mating aperture, thus comprising a bolted flange assembly.

FIG. 7A shows other cross sectional configurations of flanged joints, including the three labeled raised face, lap joint, and ring joint. It is understood that any of the four cross sections shown in FIG. 7A can be used for any of the outlet duct, inlet duct, housing outlet, or housing inlet as shown and described herein. It is understood that use of the raised face, lap joint, or ring joint in various embodiments may be facilitated by having additional features useful with the particular joint (for example, having a sealing ring to fit within the ring groove of the ring joint).

FIG. 7B shows two pairs of flange configurations, male and female. In the two figures labeled male and female, it can be seen that one flange face would include a male feature that is received within the female feature of the opposing flanged joint. Likewise, in the pair of figures labels tongue and groove it can be seen that this same idea is present in one face having a male projection that fits within a female groove on the other face. The combinations of flange faces shown in FIG. 7B include a further coupling feature which is means for aligning the duct with the housing. It is also understood that the various clamps shown in FIG. 6 include yet other features for alignment of the two faces. In FIGS. 6A, 6B, and 6C, the alignment is achieved when the periphery of the flanges is captures within the channels, the channels preferably being adapted and configured to have the inner wall of the channel pressed against the periphery of the adjoined clamps. FIG. 6D shows the use of bolts to provide alignment.

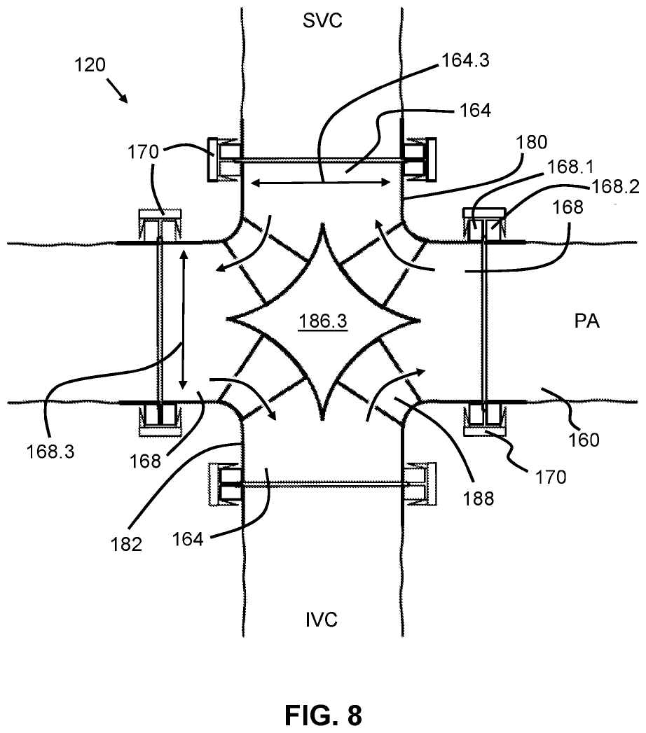

Assemblies 180 and 280 shown in FIGS. 8-11 in one embodiment are replacement components for a failed Fontan pump. They will provide a central junction for a cavopulmonary connection. These components are preferably interchangeable with the pump being replaced. It may be constructed in at least 2 ways: 1) a blank 4-way junction optimized for passive flow; 2) a 4-way junction with a central diverting body to provide optimal passive flow and minimize kinetic energy loss in the Fontan circuit. Still further embodiments pertain to 3-way junctions having 2 inlets and a single outlet.

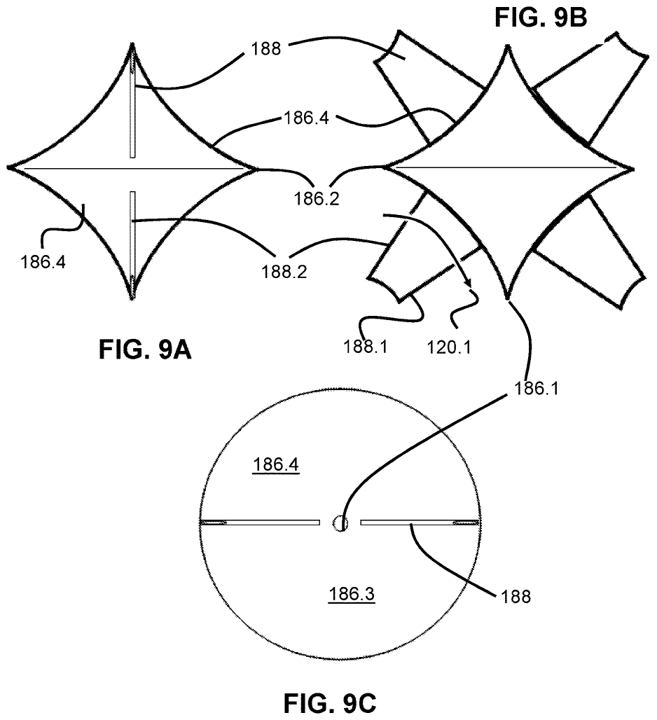

FIGS. 8 and 9 present various views of an implantable assembly 120 for passively diverting flow at a junction having multiple inlets and at least one outlet. Assembly 120 includes a replacement housing 180 preferably comprising a housing 182 having an interior, with a flow diverter 186 located by a plurality of supports struts 188 within the interior. Assembly 120 is shown located within a Fontan junction similar to the previously described. In some embodiments of the present invention, replacement housing 180 is used to replace an implantable pumping assembly 20, and is provided with an exterior shape similar to the exterior shape of the pumping assembly being replaced, and/or with an interior shape similar to the interior shape of the pumping assembly being replaced.

FIG. 8 depicts replacement assembly 180 interconnected to the Fontan junction by the use of graftable conduits 160. Preferably the conduits 160 are the same conduits that were previously attached to the anatomical structures, and previously coupled to the inlets and outlets of the pumping assembly being replaced. Pumping assembly 180 includes coupling means 164.1 at each inlet and coupling means 168.1 at each outlet. In a manner similar to that previously described, housing assembly 180 is attached to the anatomical connections 164.2 and 168.2 by the use of a plurality of clamp assemblies 170.

FIGS. 8 and 9 provide various views of the static flow diverter 186 and support struts 188 located within housing 182. Flow diverter 186 includes a body 186.3 that has an external shape similar to the pump 40 previously described. Referring to FIGS. 9A, 9B, and 9C, it can be seen that body 186.3 includes a top leading edges (points) 188.1 and smoothly curve to a common trailing edge (ring) 188.2. In some embodiments, the shape of body 186.3 provides flow surfaces 186.4 similar to the flow surfaces of a viscous impeller, and/or an overall shape similar to the overall shape of the viscous impeller. Still further embodiments contemplate a flow diverter shape similar to that of a centrifugal pump, and being either double sided about a plane of symmetry or single sided without a plane of symmetry.

However, it is understood that yet other shapes are contemplated, including one or more internal ducts or passageways that direct the internal flow, preferably directing the flow from each inlet toward each outlet. Various embodiments contemplate flow through ducts that are enclosed along their length, but open near the corresponding inlet or outlet, or having at least one side open along their length. In still further embodiments the flow surfaces of the flow diverter are adapted and configured to provide preferential diversion of flow, such as a diverter that is designed to provide more than half of the flow from a specific inlet toward a specific outlet.

Internal flow of blood within assembly 120 is naturally from higher pressure at the inlets to lower pressure at the outlets, and around the 3 dimensional central body 186.3. A simple placement of a diverting body within housing 182 can provide an acceptable internal flow field in some embodiments.

However, it is desirable to achieve an internal flow field that imparts a low pressure drop to the flowing blood. Further, it is helpful that the internal flow field between the center body and the housing interior have few or no dead zones in which blood components would be more likely to attach to the center body or the housing interior. Of further help is the establishment of an internal flow field that is generally laminar in nature.

In the embodiments shown herein, the central body of the diverter can have a shape that is similar to the 3 dimensional shape of the impeller being replaced (as shown in assembly 120), or a shape that is a 2 dimensional projection of the impeller shape (as shown inn assembly 220). However, the present invention contemplates housing interior shapes and diverter exterior shapes of any type, without reference to the shape of the replaced impeller. Preferably, such additional housings would be interconnected to the Fontan anatomy in the same manner as the pump being replaced. With such accommodation, the anatomic flanged connections would readily couple to flanged connections of the replacement housing 180 or 280.

Although the flow diverter 188 is shown and described as having a shape similar to the shape of the previously installed pump impeller, still further embodiments contemplate a diverter shape of other types. As one example, the generally concave flow surfaces could also include substantially linear portions or convex portions that assist in delivering the inflow from the inlets in a predetermined flow percentages to the outlets. Further, the flow surfaces of the diverter

In some embodiments, flow diverter 186 is statically attached to the housing by a plurality of support struts 188 that attach at an innermost end to the flow surface 186.4 and at an outermost end to the inner surface of the wall of housing 182. As shown in FIGS. 9A and 9C, one embodiment of strut 188 is generally linear from a leading edge 188.1 to a trailing edge 188.2. As depicted, strut 188 extends partly across the arc of a flow surface of the diverter body, and is located intermediate of the trailing and leading edge of the flow diverter body. Strut 188 provides limited separation and limited straightening of flow from the inlet 164 to the outlet 188.

However, the present invention contemplates various other shapes and functions for the struts. As examples, in another embodiment the struts extend more completely across the arc of the diverter flow surface, and provide increased separation and straightening of flow within the housing interior. Still further, the struts can have a curving cross sectional shape, such as the shape of an airfoil, or a more symmetrical shape with convex strut surfaces between the leading edge X88.1 and X88.2, Still further, the diverter assembly X80 can include a plurality of struts extending spherically and circumferentially around the top and bottom flow surfaces X86.4 of body X86.3, in contrast to the struts 188 shown at two (2) circumferential positions, and spaced apart by 180 degrees. Such a larger array of struts can assist in redirecting flows around the front and back sides of the housing interior that must curve around the front and back of the diverter body in order to exit. Such a larger array of flow directing struts can compensate for the 2 dimensional arrangement of inlets and outlets that provide inlet and outlet flows to the 3 dimensional shape of the diverter body.

Still further embodiments contemplate the use of flow-directing features located on the flow surface X86.4. These features can include grooves and/or ridges on the flow surface that direct the boundary layer from a particular portion of the diverter facing an inlet to a particular portion of the diverter facing an outlet.

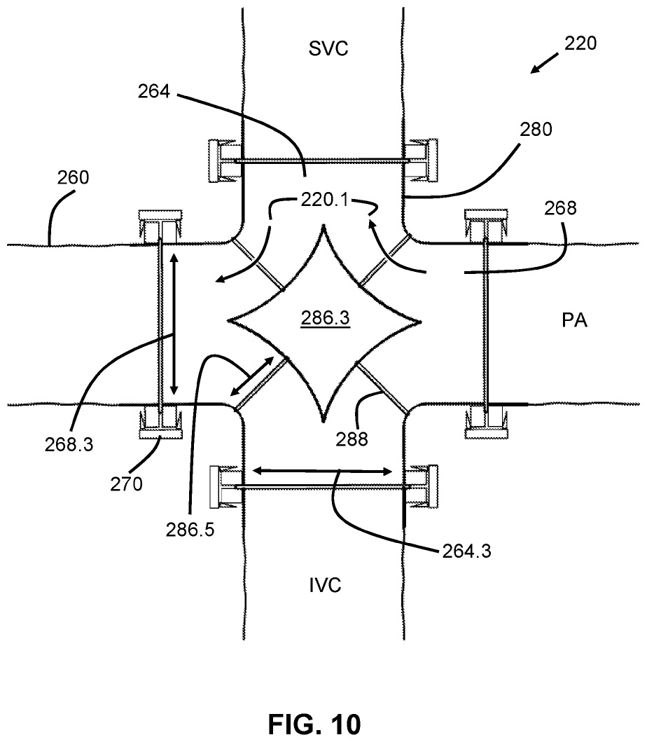

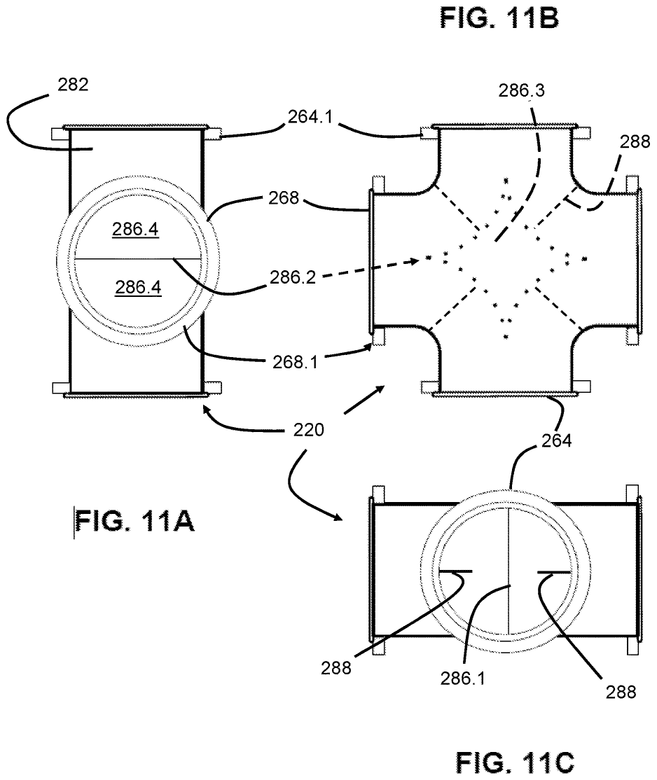

FIGS. 10 and 11 depict an implantable assembly 220 that comprises a replacement housing 280 having an interior containing a flow diverter 286 supported by a plurality of struts 288. Housing 280 is similar to housing 180, except for changes that will be described.

Struts 288 in one embodiment are preferably rod-like in cross sectional shape. Struts 288 provide an attachment of the diverter body to the housing, but provide limited straightening or redirecting of the internal flow field. Struts 288 have less surface area than struts 188, and therefore create less drag from skin friction, and also less area for the attachment of compounds carried by the blood. The use of such simpler struts is facilitated by the use of a 2 dimension interior of housing 282.

Housing 282 has an exterior shape (with corresponding interior shape) best seen in FIG. 11. Replacement housing 282 and diverter 286 create a generally 2 dimension flow field that is differently suited to the 2 dimension nature of the inlets 264 and 268 than the internal flow field within housing 182. FIGS. 11A and 11C show that the front and back sides (with reference to FIG. 11B) of the central body 286.3 are in direct contact with the corresponding front and back sides of the interior of the housing. Because of this contact at the sides, there is no opportunity for flow from the inlets to be directed in a third dimension, and not toward the outlets. This 2 dimensional correspondence of the inlets, outlets, and internal flow field can reduce or eliminate any low flow/high pressure dead or stagnant zones that could lead to deposition and accumulation of compounds carried by the blood.

In some embodiments, these front and back surfaces (the front surface being shown in FIG. 11B) provide direct attachment of the diverter body to the housing 282, in which case there may be no need for attachment struts 288. However, even if there is no need for the struts 288 to provide attachment of body to housing, struts X88 may still be useful for redirecting or straightening of the internal flow field.

Referring to FIG. 10, in some embodiments the shape of the flow field between the flow surface 286.4 of the diverter and the interior wall of the housing has a minimum cross sectional flow area 286.5 adapted and configured to provide flow with minimal pressure loss, and preferably laminar flow, from each inlet to each outlet. Each inlet 264 has a corresponding inlet flow cross sectional area 264.3, and each outlet 268 has a corresponding outlet flow cross sectional area 268.3. It is understood that the present invention contemplates assemblies X80 with any relationship between areas 264.3 and 268.3, including inlet area larger than outlet area, outlet area larger than inlet area, and generally equal inlet and outlet areas.

Preferably, the minimum cross sectional flow area 286.5 is selected to be between inlet flow area and the outlet area, or for those embodiment in which the inlet and outlet areas are the same, then about the same as the inlet and outlet areas. For the case of the inlet area being larger than the outlet area, the flow area 286.5 will provide some convergence of the internal flowpath. For the case of the inlet area being smaller than the outlet area, the flow area 286.5 will provide some divergence of the internal flowpath. Preferably, the relationship of the inlet, internal, and outlet flow areas are selected to provide low turbulence and limited or no separation of the blood boundary layer from with the diverter flow surface or the housing interior wall flow surface. In still further embodiments, the flow of fluid from inlet to outlet is generally laminar.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

1. A device for implantation in the circulatory system of an animal, comprising:
a housing comprised of a biocompatible material and having an interior and a chamber within the interior, said housing being adapted and configured for implantation within the circulatory system of an animal, said housing having first and second fluid inlets and first and second fluid outlets with each of the first and second inlets and the first and second outlets being in fluid communication with the chamber, the first and second inlets being located on generally opposite respective first and second sides of said housing, each of the first and second inlets being located in between the first and second outlets; and
a passive flow diverting member having at least one external flow surface and comprised of a biocompatible material and located centrally within the chamber, the at least one external flow surface of said passive flow diverting member being statically attached to said housing, said passive flow diverting member being adapted and configured to receive flow from each said first fluid inlet and said second fluid inlet and to passively direct flow by the at least one external flow surface to each said first fluid outlet and said second fluid outlet.

2. The device of claim 1 wherein said passive flow diverting member being adapted and configured to receive first flow from the first inlet and divert a portion of the first flow to the first outlet and the remainder of the first flow to the second outlet, and to receive second flow from the second inlet and divert a portion of the second flow to the first outlet and the remainder of the second flow to the second outlet.

3. The device of claim 1 wherein the internal passive flow diverter includes a leading edge in fluid communication with the first inlet, with flow from the first inlet on one side of the leading edge being directed to the first outlet and flow from the first inlet on the other side of the leading edge being directed to the second outlet.

4. The device of claim 1 wherein the first inlet has a flow area, and the leading edge bisects the flow area.

5. The device of claim 1 wherein the passive flow diverting member has a plane of symmetry, the first and second outlets have respective first and second flow areas, and the plane intersects each of the first and second flow areas.

6. The device of claim 1 wherein the passive flow diverting member has a plane of symmetry, the passive flow diverting member has first and second external flow surfaces on each side of the plane of symmetry, and the first and second external flow surfaces each include a corresponding first and second apex.

7. The device of claim 1 wherein said internal passive flow diverter is adapted and configured to discourage flow from the first inlet to the second inlet.

8. The device of claim 1 which further comprises a plurality of struts, each said strut connecting the interior surface of the chamber to the external flow surface of said passive flow diverting member, the external flow surface of said passive flow diverting member being spaced apart from the interior surface of the chamber by a gap sized to permit the free flow of blood through the gap.

9. The device of claim 1 wherein the biocompatible material of said housing comprises titanium or stainless steel.

10. The device of claim 1 wherein the first inlet has a first inlet flow area, said passive flow diverting member and said housing define a first pathway for the first diverted flow and a second pathway for the first remainder flow, the first pathway having a first minimum flow area, the second pathway having a second minimum flow area, and the combined first minimum flow area and the second minimum flow area are about equal to first inlet flow area.

11. The device of claim 1 wherein the first outlet has a first outlet flow area, said passive flow diverting member and said housing define a first pathway for the first diverted flow and a second pathway for the first remainder flow, the first pathway having a first minimum flow area, the second pathway having a second minimum flow area, and the combined first minimum flow area and the second minimum flow area are about equal to first outlet flow area.

12. The device of claim 1 wherein the first and second outlets being located on generally opposite respective third and fourth sides of said housing.

13. The device of claim 1 wherein the first and second inlets and first and second outlets are each adapted and configured for attachment to the circulatory system of the animal.

14. The device of claim 1 wherein the first and second inlets and first and second outlets are each adapted and configured for clamping attachment to corresponding separate flow ducts.

15. A method for directing the flow of blood in an animal, comprising:
providing a housing adapted and configured to be implantable in the circulatory system of an animal, the housing having first and second fluid inlets and first and second fluid outlets, the housing including an internal chamber and an internal flow diverter located within the internal chamber, the internal flow divider having an external flow surface statically attached to the internal chamber;
passively directing with the internal flow diverter the flow of blood from the first inlet to each of the first and second outlets; and
passively directing with the internal flow diverter the flow of blood from the second inlet to each of the first and second outlets.

16. The method of claim 15 which further comprises blocking at least some of the flow with the internal flow diverter from the first inlet to the second inlet.

17. The method of claim 15 wherein the first inlet of the housing is on one side of the chamber and the second inlet of the housing is on the opposite side of the chamber.

18. A device for implantation in the circulatory system of an animal, comprising:
a housing comprised of a biocompatible material and having an interior and an internal chamber within the interior, said housing being adapted and configured for implantation within the circulatory system of an animal, said housing having first and second fluid inlets and at least one fluid outlet, with each of the first and second inlets and the outlet being in fluid communication with the chamber, the first and second inlets being located on generally opposite respective first and second sides of said housing; and
a flow diverting member comprised of a biocompatible material and located within the internal chamber, said passive flow diverting member being statically attached to said housing, said passive flow diverting member being adapted and configured to receive flow from each said first fluid inlet and said second fluid inlet and to direct flow to the fluid outlet, said flow diverting member not including any moving parts.

19. The method of claim 18 wherein the first and second fluid inlets and said fluid outlet are each adapted and configured for attachment to the circulatory system of the animal.

20. The device of claim 18 wherein said flow diverting member includes a central diverting body located in the internal chamber.

21. The device of claim 1 wherein said housing includes a central junction and said flow diverting member includes a central diverting body located in the central junction.

22. The device of claim 1 wherein said passive flow diverting member does not include moving parts.

23. The method of claim 15 wherein the first and second fluid inlets and first and second fluid outlets are each adapted and configured for attachment to the circulatory system of the animal.

24. The device of claim 15 wherein the internal flow diverter does not include moving parts.

25. The device of claim 15 wherein said housing includes a central junction and said internal flow diverter includes a central diverting body located in the internal chamber.
